(19) United States
(12) Patent Application Publication
(10) Pub. No.: US 2018/0020948 A1
(43) Pub. Date: Jan. 25, 2018

(54) RF Attenuation Measurement System and Method

(71) Applicant: Massachusetts Institute of Technology, Cambridge, MA (US)

(72) Inventors: Ian Richard Butterworth, Cambridge, MA (US); Luca Giancardo, Dorchester, MA (US); Carlos Sanchez Mendoza, Sevilla (ES)

(73) Assignee: Massachusetts Institute of Technology, Cambridge, MA (US)

(21) Appl. No.: 15/546,426

(22) PCT Filed: Feb. 4, 2016
(86) PCT No.: PCT/US2016/016651

§ 371 Date: Jul. 26, 2017

Related U.S. Application Data
(60) Provisional application No. 62/111,922, filed on Feb. 4, 2015.

(51) Int. Cl. A61B 5/05; G01N 22/04; A61B 5/00

(52) U.S. Cl. CPC A61B 5/0507; A61B 5/4875; A61B 5/681; G01N 22/04; A61B 2503/08; A61B 2562/046

(57) ABSTRACT

In part, disclosure relates to a monitoring system for monitoring RF attenuation of a material. In one embodiment, the system includes a plurality of transmitting antennas; an RF generator having a first output terminal in electrical communication with each of the plurality of transmitting antennas and a second output terminal; a plurality of receiving antennas, each of the receiving antennas having an output terminal, and a processor having a first input terminal in electrical communication with the second output terminal of the RF generator, having a second input terminal in electrical communication with the output terminal of each of the plurality of receiving antennas, and having a first output in electrical communication with a display. In one embodiment, the processor calculates the attenuation of the RF signal in response to the RF signals received by the plurality of receiving antennas through the material.

### RELATED APPLICATIONS

This application claims priority to U.S. Provisional Application 62/111,922 filed Feb. 4, 2015, the contents of which is herein incorporated in its entirety.

### FIELD OF THE INVENTION

The invention relates to the field of radio frequency attenuation measuring devices and more specifically to devices that measure radio frequency attenuation in non-homogeneous materials in situ.

### BACKGROUND OF THE INVENTION

Radio frequency attenuation is the reduction in radio frequency (RF) signal strength as a result of the signal passing through a material. Although RF attenuation measurement devices are well known, it is difficult to measure the attenuation of an RF signal passing through a non-homogeneous non-symmetric material, such as living tissue. This is because in a non-homogeneous, non-symmetric material, the attenuation of an RF signal is determined by the electrical characteristics of the various materials through which the RF signal passes along the path from transmitting antenna to receiving antenna.

One of the reasons to measure RF attenuation through living tissue is to determine the hydration state of a human being. Adequate rehydration is a persistent and critical need in living organisms. When an individual, especially an elderly individual, becomes dehydrated, a number of symptoms and growing health concerns are likely to follow, of which some of these early symptoms, such as fatigue, weakness and loss of appetite, may promote further dehydration. To further confound the issue, these early symptoms can be subtle and easily confused by a care giver to be a common behavioural change. If dehydration persists or becomes severe, health complications can increase or become life threatening.

Hydration assessment and management is a significant challenge in clinical and care settings, especially in elderly care facilities. Eldercare homes provide live-in care for elderly residents who for many reasons might not be able to experience adequate quality of life, or receive necessary every-day or medical care in the home setting. There are more than 1.5 million residents living in more than 16,000 home care facilities. This elderly population is at risk for dehydration. This is because with old age, body water content reduces, thirst and other fluid homeostasis mechanisms degrade and the addition of increasing mental and physical frailty makes the elderly particularly vulnerable to dehydration. Studies indicate that existing monitoring regimes within care facilities fail to protect adequately residents against this vulnerability.

Although existing measures in elderly care facilities that are undertaken to prevent dehydration, such as fluid intake charts, are common and generally well-maintained, they cannot be relied upon as a sole indicator of hydration due to the lack of fluid output monitoring. This problem is particularly challenging in this typically incontinent and low-mobility population. Blood testing can provide valuable metrics for assessment of hydration level, heart and renal function, but such sample testing is mostly performed off-site, and so is restricted to high risk cases.

Whether dehydration is the primary issue, or a symptom of another condition, earlier detection and treatment of dehydration may prevent or slow the progression of various clinical decompensations, and may even be used as an early indicator of numerous common illnesses. Better monitoring for this condition is therefore desired. Because the vast majority of residents already wear a wristband for identification, some wear ankle bands for security measures, and movement sensors are also frequently utilised throughout the typical care management of the elderly resident, a mobile dehydration monitor would not impose much of an additional burden on these patients.

What is needed in general is an apparatus and method to measure RF attenuation of a signal passing through a non-homogeneous non-symmetric material. What is also needed is a wearable device that will measure the hydration of a resident and report the resident's hydration to the appropriate caregiver by measuring the attenuation of an RF signal passing through a portion of the patient.

The present invention addresses this need.

### SUMMARY OF THE INVENTION

The invention relates to an RF attenuation monitoring system for monitoring RF attenuation of a material. In one embodiment, the system includes a plurality of transmitting antennas; an RF generator having a first output terminal in electrical communication with each of the plurality of transmitting antennas and a second output terminal; a plurality of receiving antennas, each of the receiving antennas having an output terminal, and a processor having a first input terminal in electrical communication with the second output terminal of the RF generator, having a second input terminal in electrical communication with the output terminal of each of the plurality of receiving antennas, and having a first output in electrical communication with a display. In another embodiment, each of the plurality of receiving antennas is positioned to receive RF signals transmitted by each of the plurality of transmitting antennas and the RF signals received by each of the plurality of RF receivers passing through the material along one of a plurality of unique measurement paths. In yet another embodiment, the processor calculates the attenuation of the RF signal in response to the RF signals received by the plurality of receiving antennas.

In still another embodiment, each of the plurality of transmitting antennas is also a receiving antenna and each of the plurality of receiving antennas is also a transmitting antenna. In still yet another embodiment, the processor calculates a bulk RF attenuation value based on the plurality of measurement paths. In another embodiment, the plurality of transmitters and receivers are arranged to surround the material. In yet another embodiment, the plurality of transmitters and receivers are surrounded by an RF ground plane, providing partial bidirectional isolation between the material being monitored and the environment. In still another embodiment, the RF attenuation monitoring system further includes a multiplexer in electrical communication between the RF generator and each of the plurality of transmitters. In still yet another embodiment, the RF attenuation monitoring system further includes a multiplexer in electrical communication between the processor and each of the plurality of receivers.

In one embodiment, the display is in electrical communication with the processor through one of a wireless connection and a wired connection. In another embodiment, the circuit is powered by one of near field communication with an external source, an external source connected to the circuit through a wired connection, and an internal battery source. In still another embodiment, the RF generator sweeps through a plurality of frequencies. In yet another embodiment, the RF transmitting antenna is selected sequentially from the plurality of transmitting antennas. In still yet another embodiment, the RF receiving antenna is selected sequentially from the plurality of receiving antennas.

In one embodiment, the processor calculates hydration of a material in response to the attenuation of the RF signal received by the plurality of receiving antennas. In another embodiment, the processor calculates changes in hydration of a material in response to the attenuation of the RF signal received by the plurality of receiving antennas.

In part, the disclosure relates to a system of one or more computers that can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes an RF attenuation monitoring system for monitoring RF attenuation of a material, the system includes a plurality of transmitting antennas.

The RF attenuation monitoring system also includes an RF generator having a first output terminal in electrical communication with each of the plurality of transmitting antennas and a second output terminal. The RF attenuation monitoring system also includes a plurality of receiving antennas, each of the receiving antennas having an output terminal. The RF attenuation monitoring system also includes a processor having a first input terminal in electrical communication with the second output terminal of the RF generator, having a second input terminal in electrical communication with the output terminal of each of the plurality of receiving antennas, and having a first output terminal in electrical communication with a display. In one embodiment of the RF attenuation monitoring system, the plurality of receiving antennas is positioned to receive RF signals transmitted by each of the plurality of transmitting antennas and the RF signals received by each of the plurality of RF receivers passes through the material along one of a plurality of unique measurement paths.

In the RF attenuation monitoring system, the processor may calculate the attenuation of the RF signal in response to the RF signals received by the plurality of receiving antennas. Other embodiments of this aspect include corresponding computer systems, apparatuses, and computer programs recorded on one or more computer storage devices, each configured to perform the actions of the methods.

Implementations may include one or more of the following features. The RF attenuation monitoring system where the RF attenuations for each sequentially selected pair of transmitting and receiving antennas are combined over all possible transmitting antennas. The RF attenuation monitoring system where the RF attenuations at different RF frequencies, for different pairs of transmitting and receiving antennas, are combined according to a plurality of weighting values. The RF attenuation monitoring system where the weighting values are determined using an ensemble of RF attenuations obtained from an ensemble of measurements. The RF attenuation monitoring system where the weighted-combined RF attenuations at different RF frequencies and for different pairs of transmitting and receiving antennas are input to a function to output a number of dependent variables.

In one embodiment, the RF attenuation monitoring system where the function is determined from an ensemble of RF attenuations and corresponding dependent variable values as measured by any means on an ensemble of material samples. The RF attenuation monitoring system where the processor calculates hydration of a material in response to the attenuation of the RF signal received by the plurality of receiving antennas. The RF attenuation monitoring system where the processor calculates changes in hydration of a material in response to the attenuation of the RF signal received by the plurality of receiving antennas. Implementations of the described techniques may include hardware, a method or process, or computer software on a computer-accessible medium.

Implementations may include one or more of the following features. The RF attenuation monitoring system where the processor calculates changes in hydration of a material in response to the attenuation of the RF signal received by the plurality of receiving antennas. Implementations of the described techniques may include hardware, a method or process, or computer software on a computer-accessible medium.

### BRIEF DESCRIPTION OF THE DRAWINGS

The figures are not necessarily to scale, emphasis instead generally being placed upon illustrative principles. The figures are to be considered illustrative in all aspects and are not intended to limit the invention, the scope of which is defined only by the claims.

FIG. 1 is a highly schematic diagram of an embodiment of a sensor portion of a monitoring system of an embodiment of the disclosure;

FIG. 1(a) is a highly schematic diagram of an embodiment of a gain and phase detector portion of a monitoring system of an embodiment of the disclosure;

FIG. 2 is a highly schematic diagram of an embodiment of the sensor portion of an embodiment of the disclosure used for a hydration measurement;

FIG. 3 is a diagram of an embodiment of a patch antenna used in an embodiment of the disclosure;

FIG. 4 is a highly schematic diagram of an embodiment of the sensor portion of another embodiment of the disclosure in wireless communication with an embodiment of a control and display unit of the disclosure;

FIG. 5 is a highly schematic diagram of an embodiment of the sensor portion of another embodiment of the disclosure in wireless communication with another embodiment of a control and display unit of the disclosure using Near Field Communication;

FIG. 6 is a highly schematic diagram of an embodiment of the sensor portion of another embodiment of the disclosure in wireless communication with another embodiment of a control and display unit of the disclosure using a direct connection;

FIG. 7 is a cross-sectional diagram of an embodiment of the disclosure constructed as a wristband on a patient, the diagram depicting the position of the patch antennas, ground plane and electronics;

FIGS. 8(a)-(f) are the perspective, top, first end, side second end and top views of a desk hydration measurement unit constructed in accordance with the invention;

FIG. 9 is a cross-section view of the desk unit of FIG. 8;

FIGS. 10(a) and (b) depict the paths taken by signals from a transmitter to a receiver and to many receivers, respectively as used in a hydration measuring embodiment;

FIG. 11 is a flow diagram of an embodiment of data measurement steps executed by the processor of the device of the disclosure;

FIG. 12 is a flow diagram of an embodiment of the data analysis steps executed by the processor of the device of the disclosure;

FIG. 13 is a graph depicting the dielectric value and the dielectric loss for various electromagnetic frequencies;

FIG. 14 is a graph depicting the coefficient of absorption for pure and salt (sea) water plotted against frequency and wavelength; and

FIG. 15 is a schematic diagram of a multiplexer suitable for use with an embodiment of the disclosure.

### DESCRIPTION OF A PREFERRED EMBODIMENT

In brief overview, in one embodiment, the invention provides an apparatus and method for making RF attenuation measurements in a non-homogeneous and non-symmetric material. This attenuation is generally measured using RF transmitting antennas positioned on one side of the material and RF receiving antennas the other side of the material. The change in signal strength as a function of the transmitted frequency and the path from the transmitting antenna and receiving antenna through the material is then analysed by a processor to provide a measure of attenuation in the material.

One embodiment of the disclosure is a portable or wearable system for non-invasive detection of the state of hydration in a human, animal, plant, or material specimen. Hydration can refer to the state of having adequate fluid in the body tissues. The fluid is typically water or an aqueous solution. Hydration measurements are made periodically and provide an indication of the fluctuation of hydration, such as changes in fluid volume and osmolality in a specimen. Hydration is determined by measuring the attenuation of RF microwave signals as they pass through the specimen. For a human subject, the monitoring device including the associated RF transmitting and receiving antennas may be located in a wristband, ankle band, chest band, or finger ring.

In another embodiment, the sensors of the monitoring device are disposed relative to a fixed unit such as a desk, bed or table unit. The hydration measurement embodiments of the disclosure are designed to provide a way for sensitively and specifically monitoring hydration, both by the wearer (when worn by humans) and by assigned supervisors (such as caregivers). Further additional monitoring can be achieved through integration of the data into a smartphone app, website, or similar data management system, which may also provide assignable alarms for emerging significant hydration changes.

In operation, the measurement system functions by monitoring the change in amplitude and/or the phase of an RF signal (in one embodiment in the range of 0.5 to 20 GHz) that is passed through the material under test. As the signals pass along different paths through a non-homogeneous, non-symmetric material, different amounts of attenuation are measured. Similarly, in a measurement of hydration in a living being, the electromagnetic absorption characteristics will fluctuate over time as the material changes in water content percentage, osmolar concentration, and volume. The system monitors this fluctuation and transmits the information back to a data management and analysis processor to calculate and display the hydration of the wearer.

Although prior art devices do exist that measure the absorption of microwaves from a single transmitter as the microwaves pass through materials, there are significant limitations to the simplistic implementation of this method. The absorption measurement from a single RF path is highly prone to alignment and to physiologic posture changes in the case of a physiological measurement such as a hydration measurement. For instance, an implementation of this single path approach using the device in a wristband would be highly sensitive to twisting of the wrist and the subsequent change in wrist thickness and the alignment of muscle and other tissues. The present invention significantly improves this approach by adopting a multi-path RF absorption measurement and analysis approach. This permits the device to better meet the requirements, such as comfort and ergonomics, of a wearable device while minimizing inaccuracy.

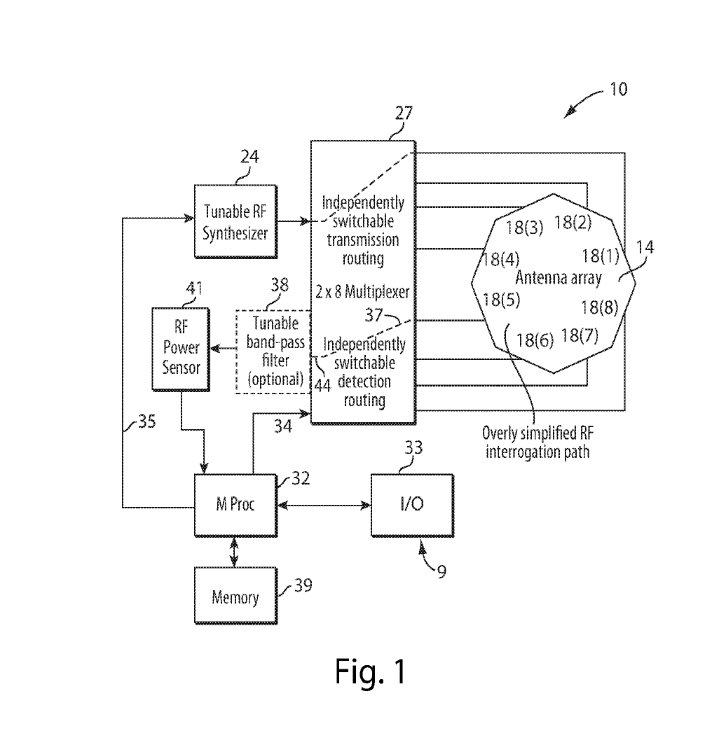

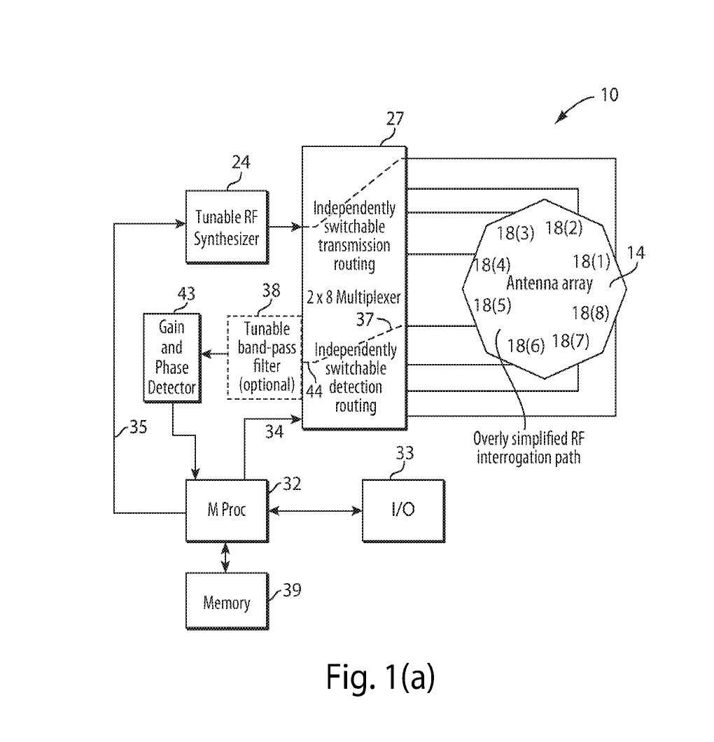

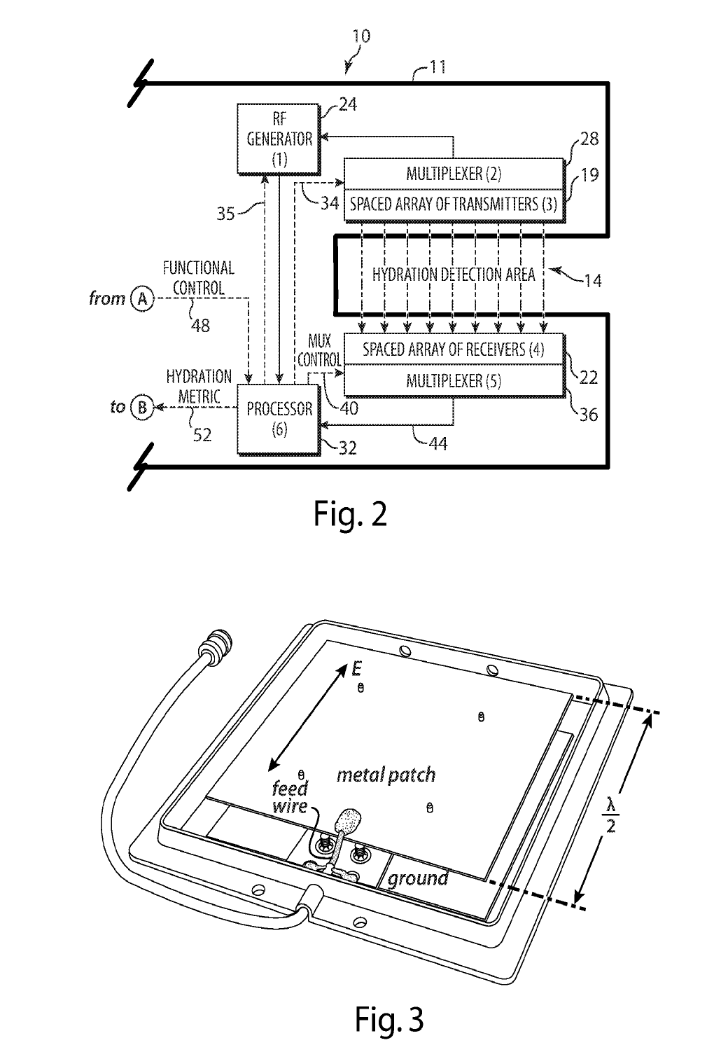

Referring to FIGS. 1, 2 and 4, in one embodiment, the system constructed in accordance with the invention includes a control and display portion 9 and a sensor portion 10. In one embodiment, the sensor portion 10 includes a housing 11 that defines an opening 14 into which the material, whose attenuation is to be measured, is placed. As shown in FIG. 2, around the opening 14 a spaced array of RF antennas 18 are positioned. In one embodiment, there are eight antennas 18(1)-18(8), but other numbers of antennas are possible. At any one time, one of the antennas 18 is acting as a transmitting antenna and the remainder are acting as receiving antennas. RF signals are transmitted from the antennas designated as RF transmitting antenna, in one embodiment for example 18(1)-18(4), pass through the material in the opening 14, and impinge on the antennas designated as RF receiving antennas 18(5)-18(8). The total number of antennas used is only constrained by the size of the antennas and the size of the wristband.

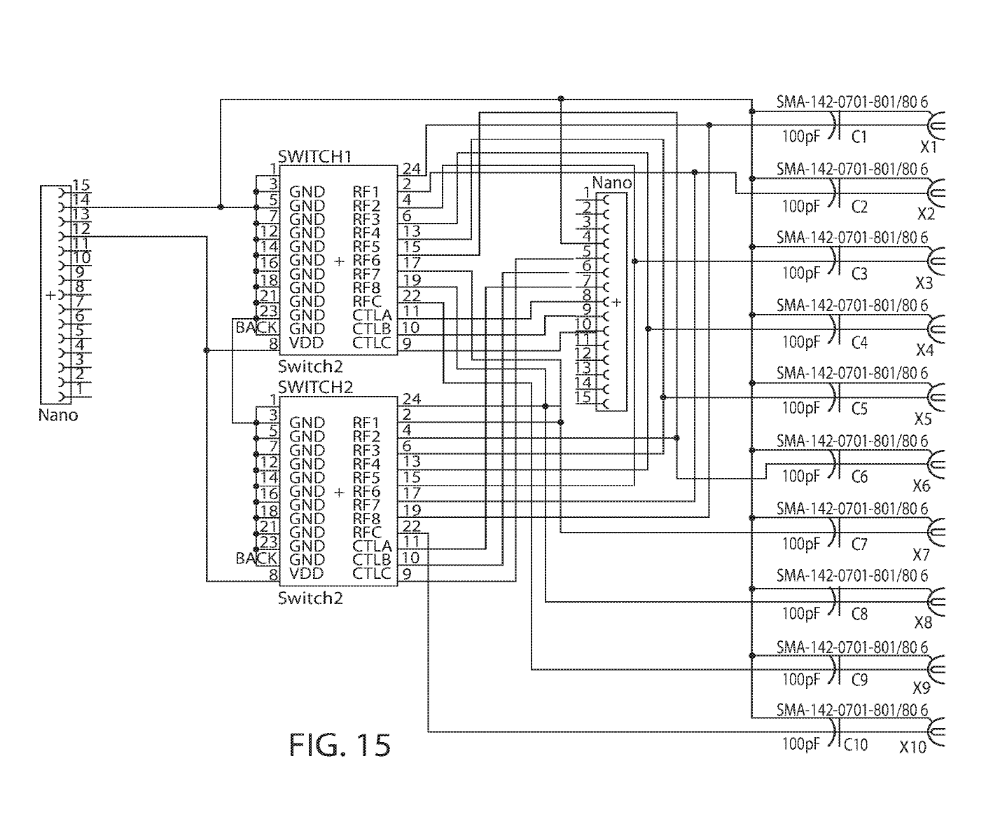

In one embodiment, a tunable RF generator (such as, for example, a SynthNV RF Generator, WindFreak Technologies LLC, Holiday, Fla.) or tunable frequency synthesizer 24 provides the RF signal for the array of RF transmitting antennas through a multichannel multiplexer 27 constructed from two RF switches such as, for example, two HMC321LP4ETR switches (Analog Devices, Norwood, Mass.) as shown in FIG. 15. In this way, the signal from the RF generator 24 can be directed to a specific RF transmitting antenna 18 by selecting which channel of the multiplexer 27 is to pass the signal from the RF generator 24 to the RF transmitting antenna 18. The selection of the multiplexer channel is performed by an output signal 34 from a processor 32 applied to a control terminal of the multiplexer 27. In one embodiment, the RF generator 24 sweeps through a range of frequencies under control of the processor connected 35 to the RF generator's control input.

Similarly, a second portion of multiplexer 27 is used by the processor 32 to select which of the antennas 18 are to act as receiving antennas. In this way, the processor 32 can select a single antenna as an RF transmitting antenna and use the remaining antennas as RF receiving antennas. This results in more signal paths through which attenuation may be determined.

Once a path has been measured, the processor 32 can then use the antenna previously designated as a transmitting antenna as a receiving antenna and one of the antennas that was previously designated as a receiving antenna as a transmitting antenna. In a configuration with eight antennas, a total of sixty-four combinations are possible. In one combination, the same antenna is used as both the transmitting antenna and the receiving antenna so as to measure back reflection. In one embodiment, the antennas are tuned to remove ambient noise sources. In one embodiment, the antennas are tuned to the center of the frequency band of the transmission frequency and a bandwidth range or wavelength selection to remove ambient noise sources.

The RF signal received from the RF receiving antenna is transmitted through the data output of the multiplexer 27 to an RF power sensor 41. In one embodiment, the RF power sensor has a frequency range of from about 1 MHz to about 4 GHz, such as, for example, an 80 dB Logarithmic Detector/Controller ADL5513 (Analog Devices, Norwood, Mass.). In one embodiment, a tunable bandpass filter 38 filters the signal from the RF receiving antenna prior to supplying the received signal to the RF power sensor 41. The tunable filter 38 is used to reduce ambient noise.

In one embodiment, the RF power sensor 41 transmits the measured power data to a data input port of the processor 32 (in one embodiment, a microcontroller ATmega32 (Atmel Corp., San Jose, Calif.)) for comparison of the relative attenuation, magnitude, and/or phase of RF frequencies passing through the material as measured against a reference attenuation ratio signal. In one embodiment, measurements are taken both at single and multiple frequencies in the RF range. In one embodiment, the RF range used is 0.5 GHz-20 GHz. In another embodiment, the preferred ranges are frequencies near 2.4 GHz and 5 GHz, as discussed in detail below.

Control of the processor 32 is accomplished using commands and data sent to the processor. In one embodiment, instructions to the processor 32 are input through a functional control signal from an I/O device 33 such as a terminal. Results of the measurements are transferred from the processor 32 to data storage or memory 39.

Referring to FIG. 1(a), the RF power sensor 41 of FIG. 1 is replaced with a gain and phase detector 43 that receives a reference signal from the tunable RF synthesizer 24 and receives a signal from the multiplexer 27 directly or through optional filter 38. The gain and phase detector provides gain and phase measurements to the microprocessor 32. The gain and phase measurements can also be used to monitor signal changes that are correlated with RF attention and/or hydration levels in a sample.

Referring to FIG. 2, in another embodiment, a portable wearable system useful in determining the hydration level of patients includes a sensor and a control portion and a separate control and display device. In one embodiment, the sensor portion 10 includes a housing 11 that defines an opening 14 to receive the body part to be measured for hydration levels. The opening 14 may be circular as in the embodiment in FIG. 1. On one side of the opening 14 is a spaced array of RF transmitting antennas 19. On the other side of the opening 14, opposite the array of RF transmitting antennas 19, is a spaced array of RF receiving antennas 22. RF signals, such as microwaves, transmitted from the RF transmitters 19 pass through the tissue positioned in the opening 14 and impinge on the antennas of the RF receivers 22.

In another embodiment, an RF generator 24 provides the signal for the array of RF transmitting antennas 19 through a multichannel multiplexer 28. In this way, the signal from the RF generator 24 can be directed to a specific RF transmitting antenna 18 by selecting which channel of the multiplexer 28 is to pass the signal from the RF generator 24 to the RF transmitting antenna 18. The selection of the multiplexer channel is performed by an output signal 34 from a processor 32 applied to the control terminal of the multiplexer 28. In one embodiment, the RF generator sweeps through a range of frequencies under control 35 of the processor 32. In this embodiment, the processor 32 is not an external computer system as shown in FIG. 1 but is embedded in the wearable unit. The I/O unit, generally 56, is a wireless device such as a tablet or smart phone as discussed below. In one embodiment, instructions to the processor 32 are input through a functional control signal 48 from an external device as described below.

A second multiplexer 36 similarly is used to control which RF receiver 22 of the spaced array of RF receivers 22 is operative to receive signals from the transmitting RF transmitter of the array 19. The second multiplexer 36 is controlled by a signal 40 also from the processor 32.

In one embodiment, both the first and second multiplexers are connected to both the transmitting antenna 19 and the receiving antennas 22 as discussed in the embodiment of FIG. 1. In this way, the processor 32 can again select a single transmitter antenna and use the remaining antennas as receiving antennas. This results in more signal paths through which attenuation may be determined. Then once a path has been measured, the processor 32 can use that transmitting antenna as a receiving antenna and, for example, one of the antennas that was previously used as a receiving antenna as a transmitting antenna as discussed previously.

The RF signal received from the RF receiver is transmitted to a data input 44 of the processor 32 for comparison of relative attenuation, magnitude, and/or phase of RF frequencies passing through the tissue with a reference attenuation ratio signal. As before, measurements are taken both at single and multiple frequencies in the RF range. As in the embodiment of FIG. 1, in this embodiment, the RF range used is from about 0.5 GHz to about 20 GHz. In another embodiment, the preferred ranges are frequencies near 2.4 GHz and 5 GHz, as discussed in detail below. Results of the measurements are transferred to the external device through a data line 52. On the other side of the opening 14, opposite the array of RF transmitters 18, is a spaced array of RF receivers 22.

Referring to FIG. 3, in each of these embodiments, the antennas of the antenna arrays in the sensor portion 10 are directional patch antennas. These patch antennas have the dimensions of a fraction of λ. λ is the corresponding wavelength of the frequency being transmitted. These small dimensions are useful in various designs. In various embodiments, these antennas are fabricated into either a rigid large enclosure, or onto flexible plastic substrates through selective metallization. The latter option is well suited to the wearable device approach given its flexibility, low cost fabrication and compact form.

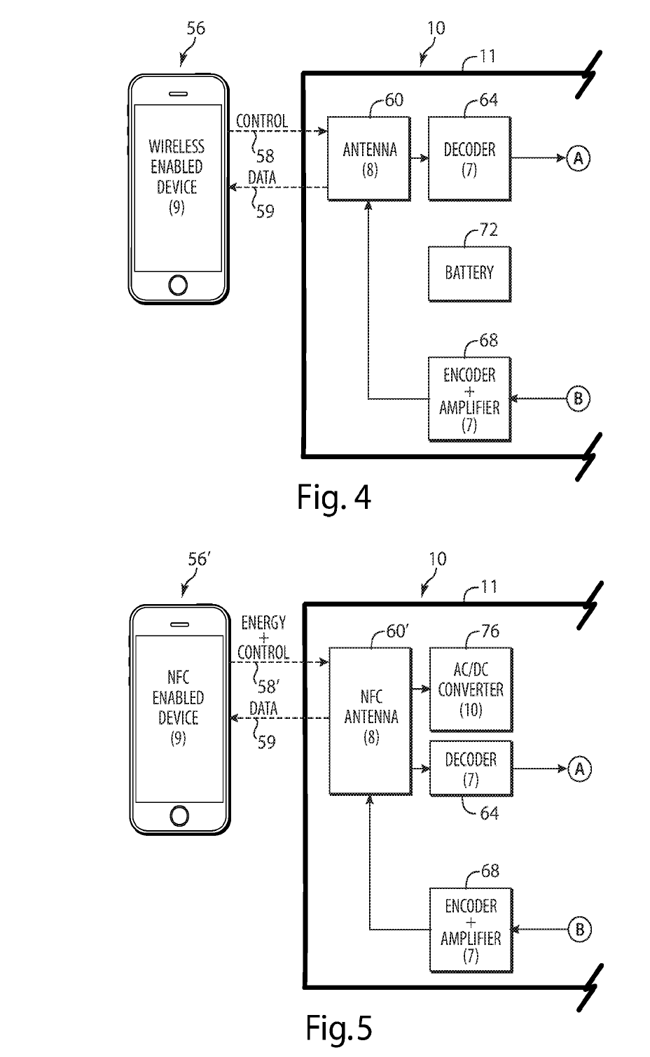

Referring also to FIG. 4, in one embodiment, especially useful in mobile devices, the sensor portion 10 of FIG. 2 communicates wirelessly with a control and display device 56/9 which is programmed to communicate with sensor portion 10. Within the sensor housing 10 is a wireless communication antenna 60 which receives commands 58 from and transmits data 59 to the control and display device 56. Signals from the communication antenna 60 are sent to a decoder 64 which converts the wireless signal protocol to functional control signals 48 (FIG. 4) that are sent to the processor 32. Similarly, data signals 52 from the processor 32 are input to an amplifier and encoder 68 that converts the data from the processor 32 to wireless protocol signals of sufficient intensity to be transmitted by the antenna 60 to the control and display device 56.

In the embodiment of FIG. 4, power is supplied to the sensor portion using a replaceable battery 72. An indicator on the sensor portion 10 notifies the user when the battery needs to be replaced.

Referring also to FIG. 5, in this embodiment, the control and display device 56′ is a Near Field Communication (NFC) device that receives data 59 from and transmits control instructions and power 58′ to the sensor portion. The antenna 60′ is an NFC antenna that sends control instructions to the decoder 64 as described previously, but sends the electromagnetic energy received to an AC to DC converter 76 which converts and uses the near field energy to power the sensor portion, thereby eliminating the need for a replaceable battery 72 (FIG. 4). In this embodiment, the device can only receive data when it is near the NFC control and display device 56′ (Broadcom NFC device family MCB2079x, Broadcom, Irvine, Calif.).

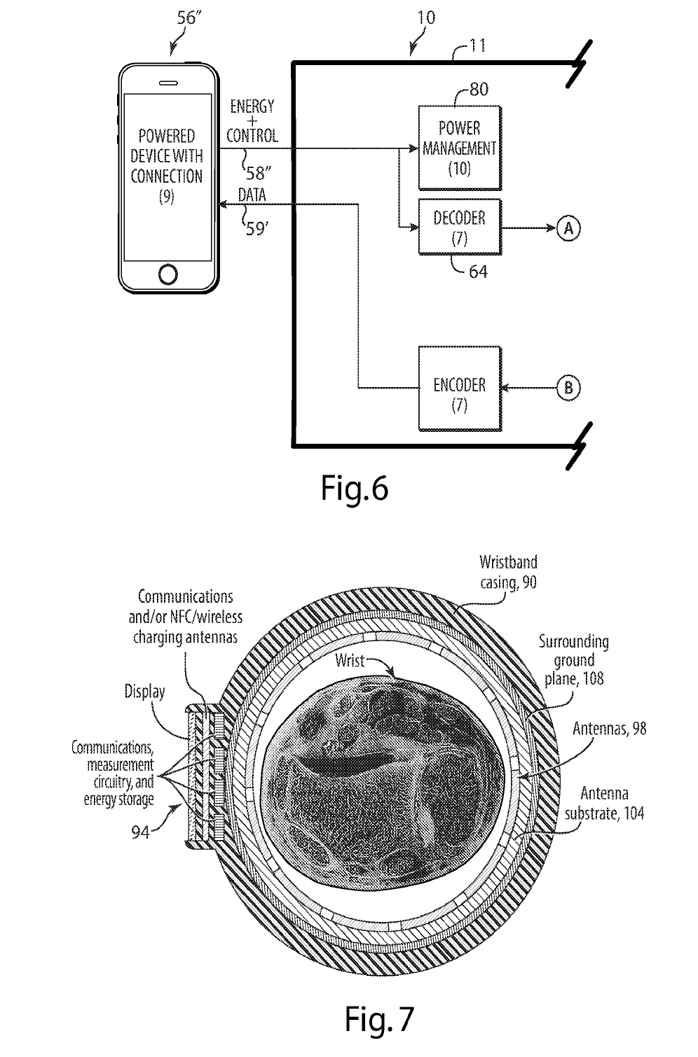

Referring also to FIG. 6, in this embodiment, the control and display device 56″ is not used wirelessly but is removably connectable to the sensor portion using a cable, for example, but not limited to, a mini-USB connector. In this embodiment, the power and control signals 58″ and the data from the sensor portion 59′ travel through the connector as separate wires within the cable. Because of the direct connection, the sensor portion 10 does not need an antenna and DC power is provided by the control and display device 56″ to a power management circuit 80 which stores the power and supplies it to the sensor portion 10 as required.

FIG. 7 provides a cross-sectional view of the device in the embodiment of a wristband for a patient. In this embodiment, the wristband includes a casing 90 to which the electronic circuits 94 of the device are mounted. Antennas 98 are positioned on an inner substrate 104 on the inner side of the band adjacent the patient's wrist. A ground plane is positioned between the antennas 98 and the outer side of the band.

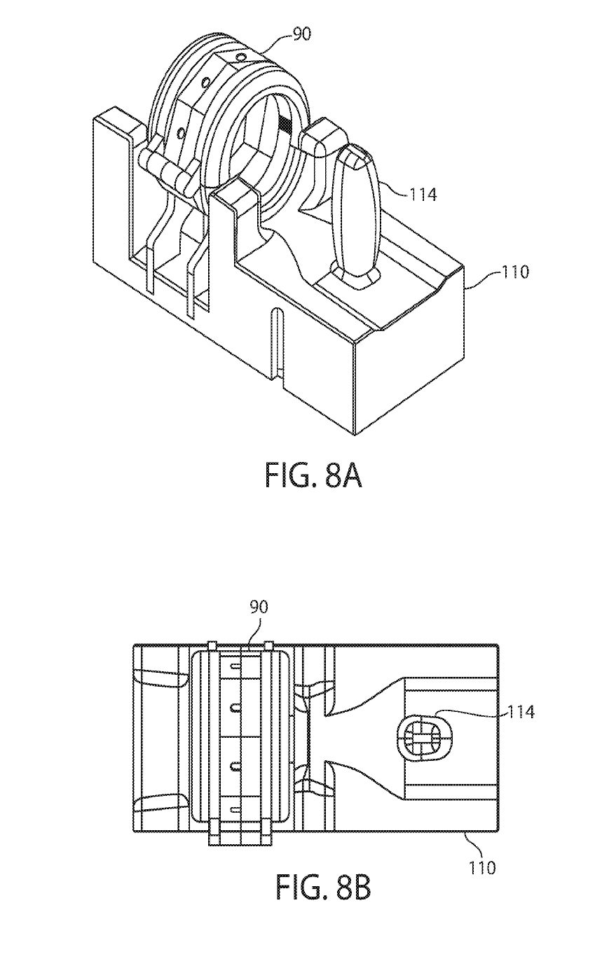

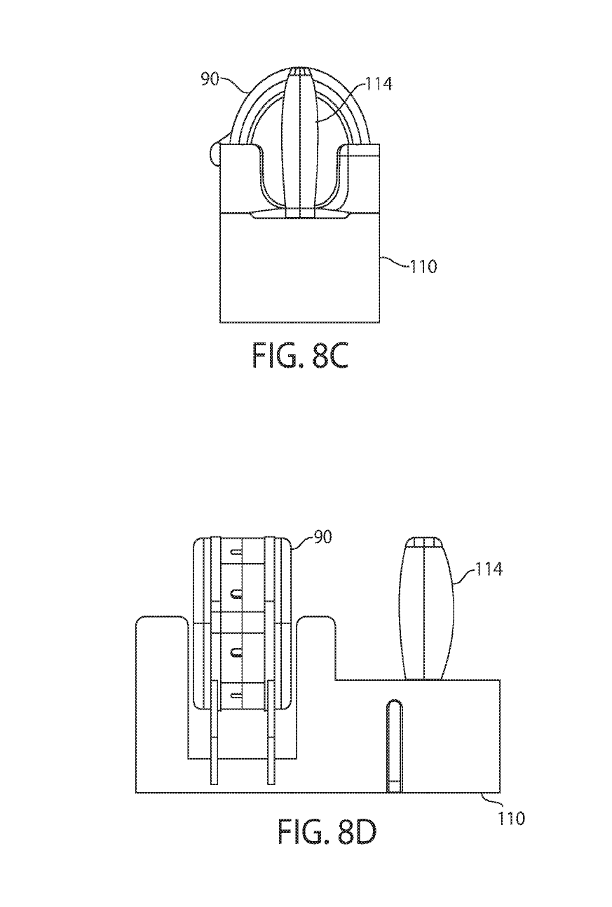

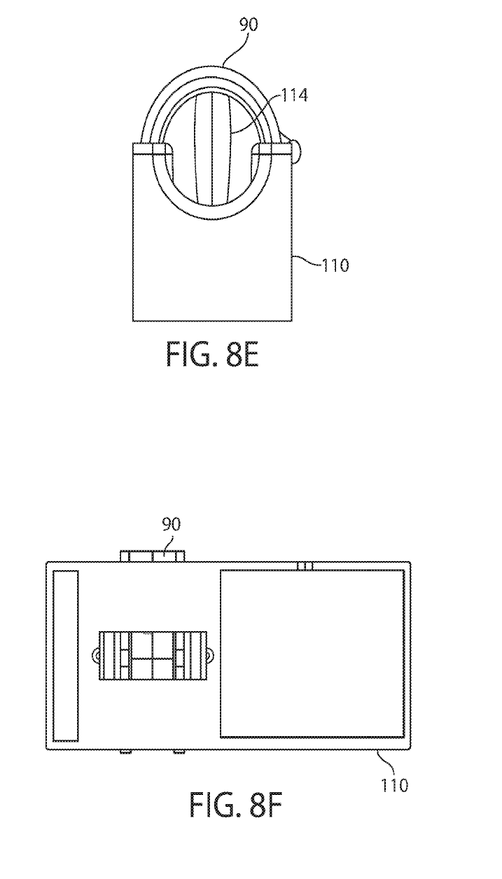

FIGS. 8 depict the perspective 8(a), top 8(b), first end 8(c), side 8(d), second end 8(e) and bottom 8(f) views of a desk unit for measuring hydration. In this unit, the wristband housing 90 is mounted on the stand 110. A handle 114 is also mounted on the stand 110 in line with the center of the wristband 90. The user places his or her arm within the wristband 90 and grasps the handle 114, to assure proper alignment of the wrist with the RF antennas. The base may either have its own display and key pad or simply be connectable to a computer through a wire or wirelessly.

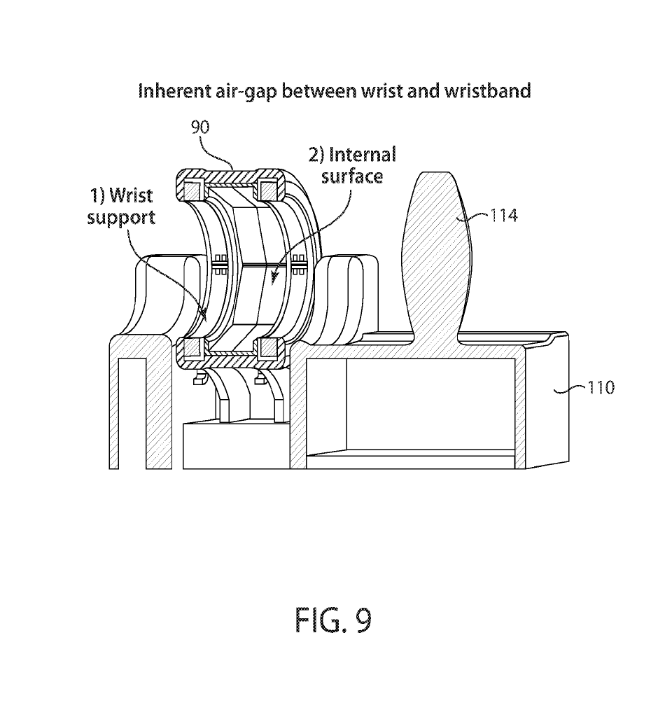

FIG. 9 depicts a cross-section view of the device of FIG. 8, showing the wrist support bands 120 and the surfaces of the patch antennas 130, showing how they are recessed and do not make contact with the arm of the user. In one embodiment, the control and display modules are located in an external device or otherwise disposed external to a housing of the desk unit. In one embodiment, the control and display modules are located in the desk unit itself.

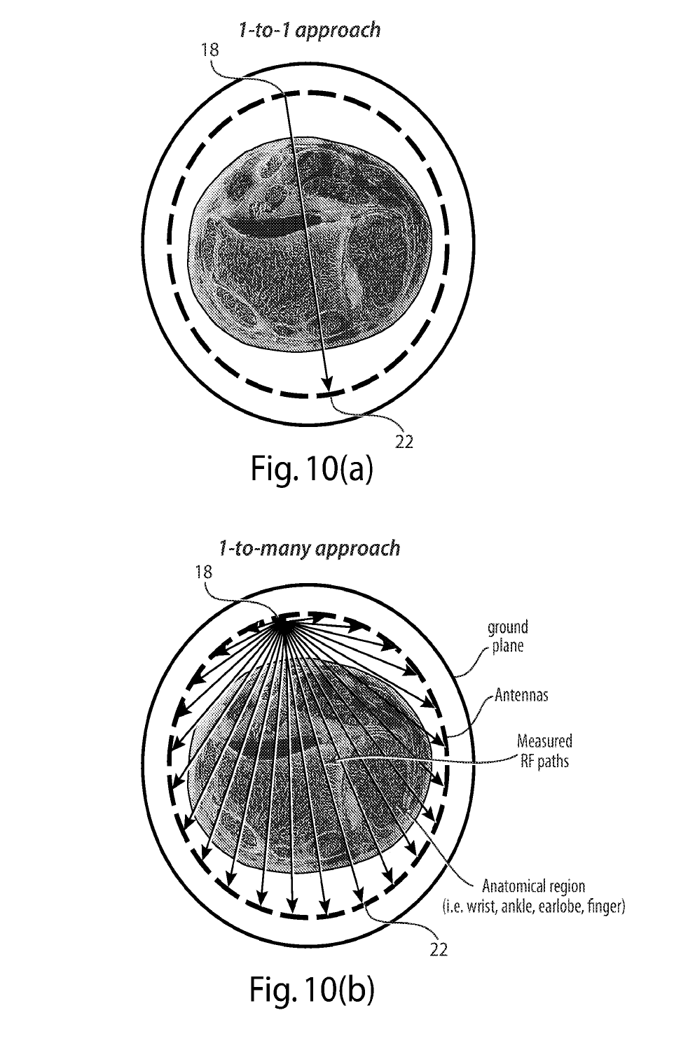

In operation and referring to FIG. 10(a), in one embodiment, the sensor portion is used in a one-to-one configuration in which a signal from the transmitting antenna is received by one specific receiver antenna. The attenuation, phase and power of the signal are therefore measured along a single line through the material being measured. In a second embodiment (FIG. 10(b)), the configuration is one-to-many wherein the signal being transmitted by the transmitting antenna is received by multiple receiving antennas simultaneously. In this embodiment, the attenuation, phase and power of the signal being transmitted by the transmitting antennas are therefore measured along multiple paths through the material being measured.

Whether used generally in the measurement of RF attenuation in a material or specifically in determining changes in hydration in a human, the processor 32 processes the received signal and makes use of the fact that transmission of common communication-range RF frequencies through a material is highly sensitive to the electrical characteristics of the material in the path of the signal. In the case of a hydration measurement device, the attenuation is sensitive to both amount of water and the fluid osmolality of the tissues in the signal path. In this way, the wearable device provides measurements of the volume and/or osmolality changes for the wearer.

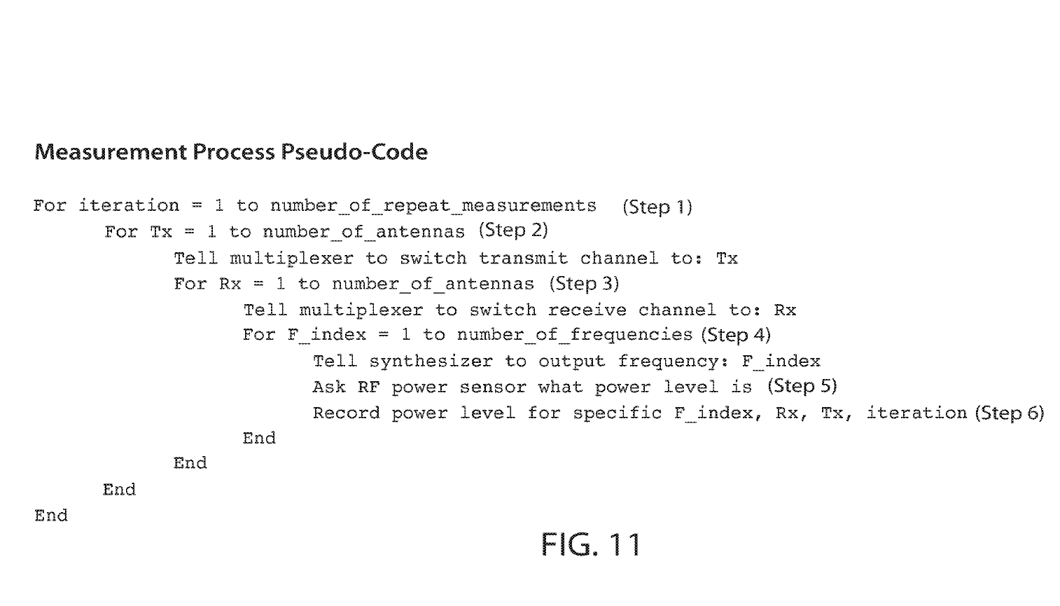

Regardless of whether the measurement made by the system is general RF attenuation or specifically RF attenuation to determine hydration, the steps executed by the processor 32 to control the measurement of attenuation are the same in one embodiment. An exemplary embodiment of method steps to monitor RF attenuation and generate data corresponding to or correlated with hydration levels is shown in FIG. 11.

Referring to FIG. 11, a set of method steps are iteratively performed for different antennas and signals as a series of nested processing loops. Generally, the order is first to determine how many times the measurements are to be repeated (Step 1). Next, the processor 32 selects which antenna is to be the transmitting antenna (Step 2). The transmitting antenna is incremented using a counter to track this in one embodiment. This can also be part of Step 2 or performed separately. Selecting which antenna is to be the receiving antenna (Step 3) is performed. This can also be tracked with a counter. Selecting the frequency index of the frequency of the RF signal to be transmitted is performed (Step 4). In addition, determining the power level for that frequency (Step 5) is performed. The power value for the transmitting antenna, receiving antenna, frequency index and iteration is stored in a four-dimensional received power matrix (Step 6). This matrix is subsequently transformed by reducing its dimensionality.

In one embodiment, the frequency of the RF signal is incremented (Step 4) using the same transmitting and receiving antennas and the measurement steps 5 and 6 are repeated. This is repeated until all the frequencies to be tested are used. At this point, the RF frequency is reset to the initial frequency index value and the next receiving antenna is selected (Step 3). Again, the power level is determined (Step 5) and the data stored as a power matrix (Step 6). This frequency incrementing loop is then incremented using the same transmitting and receiving antennas, and the measurement steps 5 and 6 are repeated until the received power for all the frequencies are measured for the receiving antenna. The next receiving antenna is then selected and the frequency loop is repeated for each frequency of interest. The frequency loop is repeated for each receiving antenna until all the receiving antennas have been used to measure all the frequencies of interest.

As an overview, the frequency and receiving antenna loops are repeated for each transmitting antenna. Once all the transmitting antennas are utilized, the frequency index, receiving antenna, and transmitting antenna are reset to their initial values and the iteration number is incremented. As noted above, this process results in incremental measurements over time and one or more power matrices as the output data. Once power for the set of applicable frequencies has been measured for all the receiving antennas, the frequency is returned to the initial frequency and the receiving antenna is reset to the initial value.

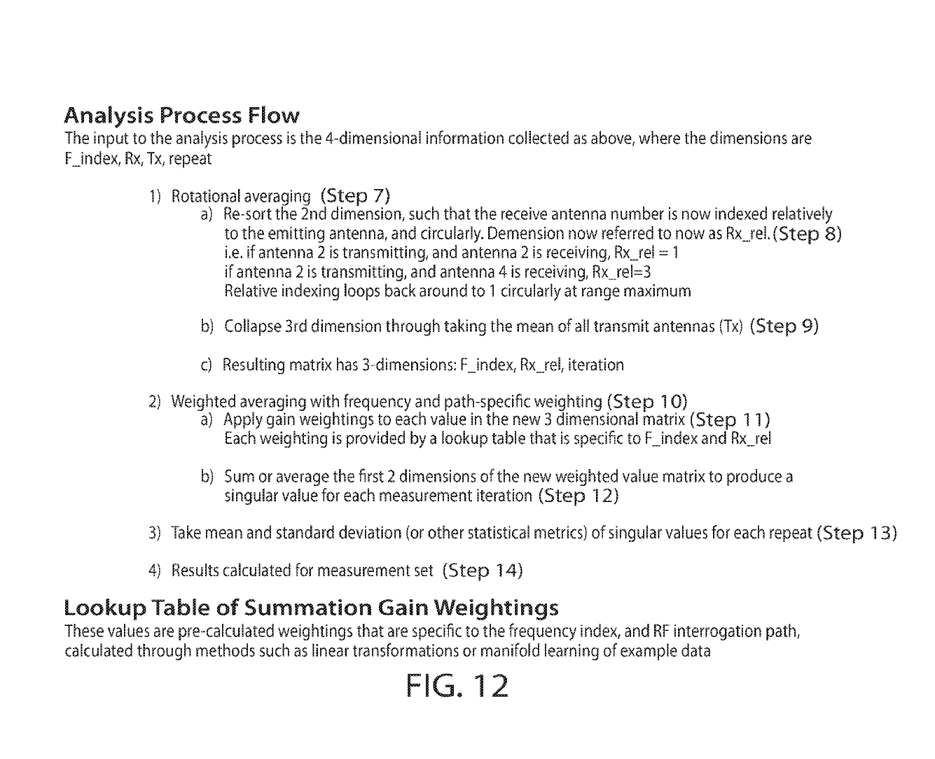

Once a received power matrix has been populated with data, the processor 32 can analyze the data. Referring to FIG. 12, the processor 32 performs the attenuation analysis using the power matrix and/or its underling data. The analysis process begins by performing rotational averaging of the received power matrix to form a rotational averaged received power matrix (Step 7). This averaging begins by sorting the received power matrix such that the receiving antenna number is indexed relative to which antenna is the transmitting antenna (Step 8). This index is termed Rx_rel. For example, if antenna 2 is both transmitting and receiving antenna, then Rx_rel=1. If antenna 2 is transmitting and antenna 3 is receiving, then Rx_rel=2. If antenna 2 is transmitting and antenna 4 is receiving, then Rx_rel=3, and so on. This process is repeated for each antenna.

Next, the power matrix is averaged over the transmitting antenna to obtain a rotational averaged received power matrix. Next, the mean power of all the transmitting antennas is determined (Step 9). This step 9 can include collapsing the transmitting antenna dimension of the received power matrix to a single value. This step causes the power matrix, after rotational averaging, to have only three dimensions: frequency index, Rx_rel, and iteration number.

Next, using the processor 32, weighted averaging with frequency weighting and/or path-specific weighting is performed (Step 10). This step 10 can include determining specific weights for the different frequency and Rx_rel values from training data in order to maximize the variability of the weighted rotational averaged received power matrix over different samples in the training data (Step 11). Thus, step 11 applies gain weightings to each value in the new three dimensional matrix. In one embodiment, each weighting is provided by a lookup table that is specific to F_index and Rx_rel. These values are pre-calculated weightings that are specific to the frequency index and RF interrogation path in one embodiment. These values can be calculated through methods such as linear transformations or manifold learning of example data.

As part of the weighted averaging process, (Step 10), summing or averaging the first 2 dimensions of the new weighted value matrix can be performed to produce a singular value for each measurement iteration (Step 12). The method can also include using weighting values by maximizing the variability of the resulting weighted rotational averaged received power matrix for a set of training data corresponding to different hydration statuses. The method can include applying the weights to a given test rotational averaged received power matrix instance, to obtain a weighted rotational averaged received power matrix.

In one embodiment, the resulting weighted rotational averaged received power matrix is used as input to a regression function. In one embodiment, the function maps such a weighted rotational averaged received power matrix into a one- or multi-dimensional dependent variable. The regression function is determined using weighted rotational averaged received power matrix from training data involving different hydration status and corresponding values of the sought dependent variable. In one embodiment, the regression function used to generate outputs is computed using a collection of weighted rotational averaged received power matrix from training data corresponding to different hydration status and corresponding values of the uni- or multi-dimensional sought dependent variable.

In addition, the method can include generating a mean and standard deviation (or other statistical metrics) of singular values for each iteration/repeated measure (Step 13). The method can include applying the regression function to the given test rotational averaged received power matrix instance to compute a dependent uni- or multi-dimensional dependent variable, which constitutes the output of the system. As a next step, (Step 14), results are calculated using the measure and determined values to generate hydration values/levels. This step can include applying data to the regression function. These results can be displayed using one or more user interfaces on various types of output devices. In one embodiment, the output device is a watch or band or bracelet or other device or system that monitors hydration or otherwise logs physiological data.

The attenuation of the material measured is most easily explained by considering the measurement of hydration. The sensitivity of the RF transmission through water is a result of the fact that liquid water has a broad absorption spectrum in the RF microwave region. This in turn can be explained in terms of changes in the hydrogen bond network, and these changes give rise to a broad, featureless, microwave absorption spectrum. This absorption of microwave energy is equivalent to dielectric loss and is what causes food that contains water molecules to heat in a microwave oven. Typically, microwaves with a frequency of 2.45 GHz, wavelength 122 mm, are used in a microwave oven. This frequency is also shared by most modern wireless communication standards, such as Wi-Fi (2.4 and 5 GHz) and Bluetooth (2.4 GHz).

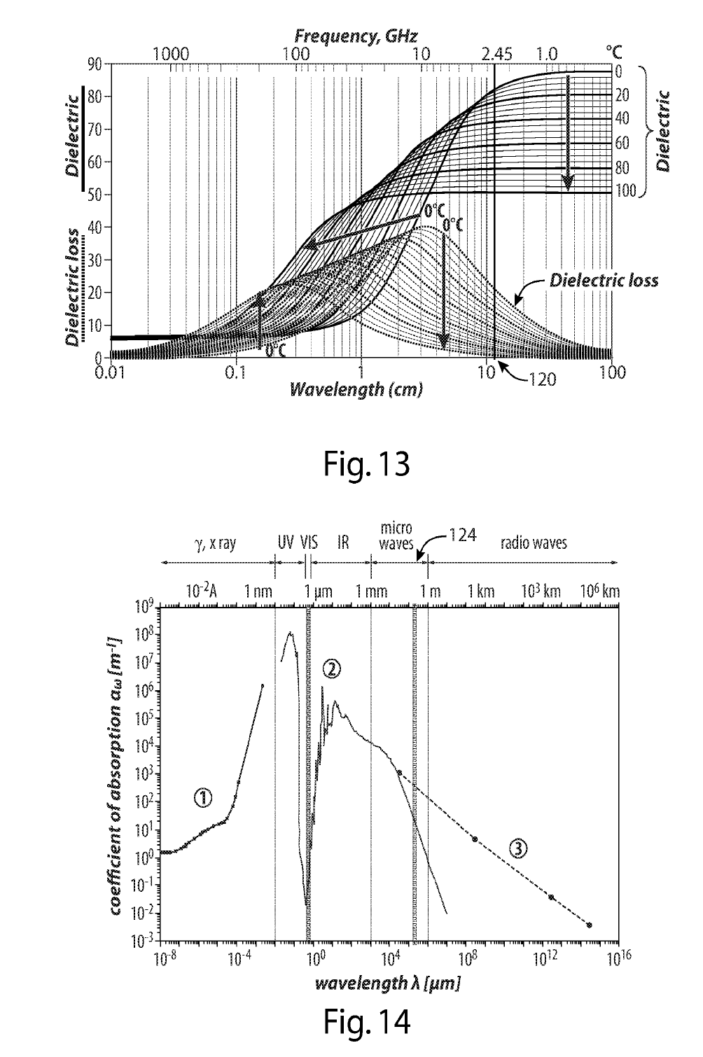

Referring to FIG. 13, the dielectric permittivity and dielectric loss of pure water between 0° C. and 100° C. is shown, with a broad vertical line 120 indicating the effect of increasing temperature at 2.45 GHz. For pure water alone, this absorption characteristic demonstrates sensitivity to path length between the transmitter and the receiver. Further, the lower GHz range for RF absorption coefficients of water are affected by salinity, which attenuates typical GHz communication frequencies much more than that of pure water. Thus, a sensitive measurement of changing osmolality can be made using the systems and methods described herein. In one embodiment, the device can alarm or generate an alert if hydration levels are below a threshold. In this way, dehydration of athletes, the elderly, and others can be avoided.

Referring to FIG. 14, the absorption spectra of pure water (1) and (2), and salt water (sea water concentration) (3) is shown with the 2.4 GHz frequency range of microwaves indicated. The difference between the intersection of 2.4 GHz (arrow indicated vertical line 124), and the pure water (solid) and salt (sea) water (dotted) lines, provides a good indicator for the variation in absorption coefficient due to salinity at lower GHz frequencies. Because of this change in absorption in response to electrolytic concentration, a transmission measurement in this lower frequency microwave range will be sensitive to fluctuations in the length of the transmission path, volume of water, and the proportion of electrolytes within this fluid volume. By conducting both measurements at this low GHz frequency range, where electrolytic content of water contributes significantly to dielectric loss, and higher GHz frequencies, where losses due to the water dominate, an indication of water and electrolytic content may be determined. The dominance of RF losses due to water above the low GHz range (above 3 GHz) means that the attenuation of RF signals is an indicator of the amount of water present in the transmission path through the tissue.

In one embodiment, the RF power sensor may be replaced by an RF gain and phase detector (AD8302 RF Gain and Phase Detector, Analog Devices, Norwood Mass., USA) to provide both gain and phase of the detection path relative to the RF synthesizer output. This measurement of gain and phase provides a measurement of complex impedance, allowing the determination of the reactive component of the dielectric loss mechanism in the material, as well as the resistive counterpart. This complex impedance allows the determination of a dielectric loss of the bulk material.

Some portions of the detailed description are presented in terms of algorithms and symbolic representations of operations on data bits within a computer memory. These algorithmic descriptions and representations can be used by those skilled in the computer and software related fields.

The algorithms and displays presented herein are not inherently related to any particular computer or other processor apparatus provided it is capable of transmitting and receiving data. Various general purpose systems may be used with programs in accordance with the teachings herein, or it may prove convenient to construct more specialized apparatus to perform the required method steps. The required structure for a variety of these systems will appear from the description below. In addition, the present invention is not described with reference to any particular programming language, and various embodiments may thus be implemented using a variety of programming languages.

Unless otherwise indicated, all numbers expressing lengths, widths, depths, or other dimensions and so forth used in the specification and claims are to be understood in all instances as indicating both the exact values as shown and as being modified by the term “about.” As used herein, the term “about” refers to a ±20% variation from the nominal value. Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Any specific value may vary by 20%.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

In one or more example embodiments, the functions and methods described may be implemented in hardware, software, or firmware executed on a processor, or any combination thereof. If implemented in software, the functions may be stored on or transmitted over as one or more instructions or code on a computer-readable medium or memory. Computer-readable media include both non-transitory computer storage media and communication media including any medium that facilitates transfer of a computer program.

A storage medium may be any available media that can be accessed by a computer. By way of example, and not limitation, such computer-readable media can include non-transitory computer-readable media including RAM, ROM, EEPROM, CD-ROM or other optical disk storage, magnetic disk storage or other magnetic storage devices, or any other medium that can be used to carry or store desired program code in the form of instructions or data structures and that can be accessed by a computer. A computer-readable medium can include a communication signal path. For example, if the software is transmitted from a website, server, or other remote source using a coaxial cable, fiber optic cable, twisted pair,), or wireless technologies such as infrared, radio, and microwave, then the coaxial cable, fiber optic cable, twisted pair, DSL, or wireless technologies such as infrared, radio, and microwave are included in the definition of medium.

The system may include various modules as discussed above. As can be appreciated by one of ordinary skill in the art, each of the modules may include one or more of a variety of sub routines, procedures, definitional statements and macros. Each of the modules may be separately compiled and linked into a single executable program. Therefore, the description of each of the modules is used for convenience to describe the functionality of the disclosed embodiments. Thus, the processes that are undergone by each of the modules may be redistributed to one of the other modules, combined together in a single module, or made available in, for example, a shareable dynamic link library.

The system may be used in connection with various operating systems such as Linux, or Microsoft Windows, Android, OS and others. The system may be written in any conventional programming language such as C, C++, BASIC, Pascal, or Java, and ran under a conventional operating system. The system may also be written using interpreted languages such as Visual Basic (VB.NET), Perl, Ruby, or Python.

It will be appreciated by those skilled in the art that various modifications and changes may be made without departing from the scope of the described technology. Such modifications and changes are intended to fall within the scope of the embodiments that are described. It will also be appreciated by those of skill in the art that features included in one embodiment are interchangeable with other embodiments; and that one or more features from a depicted embodiment can be included with other depicted embodiments in any combination. For example, any of the various components described herein and/or depicted in the figures may be combined, interchanged, or excluded from other embodiments.

What is claimed is:

1. An RF attenuation monitoring system for monitoring RF attenuation of a material, the system comprising:
a plurality of transmitting antennas;
a RF generator having a first output terminal in electrical communication with each of the plurality of transmitting antennas and a second output terminal;
a plurality of receiving antennas, each of the receiving antennas having an output terminal, and
a processor having a first input terminal in electrical communication with the second output terminal of the RF generator, having a second input terminal in electrical communication with the output terminal of each of the plurality of receiving antennas, and having a first output in electrical communication with a display,
wherein each of the plurality of receiving antennas is positioned to receive RF signals transmitted by each of the plurality of transmitting antennas and the RF signals received by each of the plurality of RF receivers passing through the material along one of a plurality of measurement paths, and
wherein the processor calculates the attenuation of the RF signal in response to the RF signals received by the plurality of receiving antennas.

2. The RF attenuation monitoring system of claim 1 wherein each of the plurality of transmitting antennas is also a receiving antenna and wherein each of the plurality of receiving antennas is also a transmitting antenna.

3. The RF attenuation monitoring system of claim 1 wherein the processor calculates a bulk RF attenuation value based on the plurality of measurement paths.

4. The RF attenuation monitoring system of claim 1, wherein the plurality of transmitters and receivers are arranged to surround the material.

5. The RF attenuation monitoring system of claim 1, where the plurality of transmitters and receivers are surrounded by an RF ground plane, providing partial bidirectional isolation between the material being monitored and the environment.

6. The RF attenuation monitoring system of claim 1, further comprising a multiplexer in electrical communication between the RF generator and each of the plurality of transmitters.

7. The RF attenuation monitoring system of claim 1, further comprising a multiplexer in electrical communication between the processor and each of the plurality of receivers.

8. The RF attenuation monitoring system of claim 1, wherein the display is in electrical communication with the processor through one of a wireless connection and a wired connection.

9. The RF attenuation monitoring system of claim 1, wherein the circuit is powered by one of near field communication with an external source, an external source connected to the circuit through a wired connection, and an internal battery source.

10. The RF attenuation monitoring system of claim 1, wherein the RF generator sweeps through a plurality of frequencies.

11. The RF attenuation monitoring system of claim I, wherein the RF transmitting antenna is selected sequentially from the plurality of transmitting antennas.

12. The RF attenuation monitoring system of claim 1, wherein the RF receiving antenna is selected sequentially from the plurality of receiving antennas.

13. The RF attenuation monitoring system of claim 1, wherein the processor calculates hydration of a material in response to the attenuation of the RF signal received by the plurality of receiving antennas.

14. The RF attenuation monitoring system of claim 13, wherein the processor calculates changes in hydration of a material in response to the attenuation of the RF signal received by the plurality of receiving antennas.

15. The RF attenuation monitoring system of claim 1, wherein the RF attenuations for each sequentially selected pair of transmitting and receiving antennas are combined over all possible transmitting antennas.

16. The RF attenuation monitoring system of claim 1, wherein the RF attenuations at different RF frequencies, for different pairs of transmitting and receiving antennas, are combined according to a plurality of weighting values.

17. The RF attenuation monitoring system of claim 16, wherein the weighting values are determined using an ensemble of RF attenuations obtained from an ensemble of measurements.

18. The RF attenuation monitoring system of claim 17, wherein the weighted-combined RF attenuations at different RF frequencies and for different pairs of transmitting and receiving antennas are input to a function to output a number of dependent variables.

19. The RF attenuation monitoring system of claim 1, wherein the function is determined from an ensemble of RF attenuations and corresponding dependent variable values as measured by any means on an ensemble of material samples.

20. The RF attenuation monitoring system of claim 1, wherein the processor calculates hydration of a material in response to the attenuation of the RF signal received by the plurality of receiving antennas.

21. The RF attenuation monitoring system of claim 13, wherein the processor calculates changes in hydration of a material in response to the attenuation of the RF signal received by the plurality of receiving antennas.
